(19) United States
(12) Patent Application Publication
(10) Pub. No.: US 2020/0294629 A1
(43) Pub. Date: Sep. 17, 2020

(54) GENE SEQUENCING DATA COMPRESSION METHOD AND DECOMPRESSION METHOD, SYSTEM AND COMPUTER-READABLE MEDIUM

(71) Applicant: GENETALKS BIO-TECH (CHANGSHA) CO., LTD., Hunan (CN)

(72) Inventors: Zhuo SONG, Hunan (CN); Gen LI, Hunan (CN); Zhenguo WANG, Hunan (CN); Bolun FENG, Hunan (CN); Haibo MAO, Hunan (CN); Xiali XU, Hunan (CN); Chouxian MA, Hunan (CN)

(73) Assignee: GENETALKS BIO-TECH (CHANGSHA) CO., LTD., Hunan (CN)

(21) Appl. No.: 16/618,401

(22) PCT Filed: Sep. 18, 2018
(86) PCT No.: PCT/CN2018/106188

§ 371 Date: Dec. 2, 2019

(30) Foreign Application Priority Data
Oct. 24, 2017 (CN) 201710999663.2

(51) Int. Cl. G16B 50/50; G16H 10/40; H03M 7/30

(52) U.S. Cl. CPC G16B 50/50; H03M 7/30; G16H 10/40

(57) ABSTRACT

The invention discloses a gene sequencing data compression method and decompression method, a system, and a computer-readable medium. The compression method includes: comparing a read sequence R with a reference genome to obtain an equal-length gene character sequence CS; coding the read sequence R and the equal-length gene character sequence CS, performing reversible computing by means of a reversible function, compressing a most approximate position p of the read sequence R in the reference genome and the reversible computing result that serve as two data streams, and outputting the compressed data streams. The data decompression method is reverse processing of the compression method. By means of the present invention, the compression ratio can be further decreased, the compression/decompression time of an algorithm is shorter while a better compression ratio is obtained. The present invention is compatible with algorithms for making comparisons between read sequences and reference genomes.

### TECHNICAL FIELD

The present invention relates to gene sequencing and data compression technologies, in particular to a gene sequencing data compression method and decompression method, a system, and a computer-readable medium.

### BACKGROUND

As next generation sequence (NGS) keeps unfolding in recent years, gene sequencing is fast in speed and low in cost. Moreover, the gene sequencing technology has been extensively popularized and applied in various fields of biology, medicine, heath, criminal investigation, agriculture, etc., which leads to the explosive growth in original gene sequencing data at 3˜5 times every year, even faster. Besides, every gene sequencing sample is very big, for example, one person's 55 x whole genome sequencing data is about 400 GB. Hence, there are technology and cost challenges for storage, management, retrieval and transmission of massive gene testing data. Data compression is one of technologies to mitigate this challenge. Also, it is a process of converting data to be more compact than original format data to decrease the storage space. Original input data comprises a symbol sequence to be compressed or reduced. These symbols are coded by a compressor and output as coded data. At some later time point, the coded data is generally input into a decompressor to be decoded and rebuilt, and then the original data is output in a symbol sequence way. If the output data is always identical to the input data completely, this compression scheme is lossless, also called a lossless encoder. Otherwise, it is a lossy compression scheme.

At present, researchers from various countries in the world have developed various gene sequencing data compression methods. Based on applications thereof, the gene sequencing data compressed must be rebuilt and restored to be original data whenever possible. Hence, the gene sequencing data compression methods with actual meanings refer to lossless compression. In case of classifying based on the total technical route, the gene sequencing data compression method may be divided into general purpose, reference-based and reference-free compression algorithms.

The reference-based compression algorithm comprises the steps of selecting a certain genome data as a reference genome, and indirectly compressing data using features of the gene sequencing data and the similarity between target sample data and reference genome data. Common similarity representation, coding and compression methods of the existing reference-based compression algorithms mainly comprise Huffman coding compression algorithm, dictionary method represented by LZ77 and LZ78, arithmetic coding compression algorithm, and other basic compression algorithms and their variant and optimization compression algorithms. For human beings, the reference genome has almost 3 GB A/C/G/T characters. For this, every read sequence of gene sequencing data obtained by sequencing is matched to a certain position of this 3 GB character string. Based on the above features, in the reference-based compression algorithm of the prior art, if a certain read sequence is compared to a certain position of the reference genome, it is depicted by position information of one relative reference genome and one cigar string. On account that most read sequences are not completely matched with a reference sequence, the cigar string is generally like this: for example, the reading sequence is “ . . . ACCTTGG . . . ”, the matched reference sequence of which in the reference genome is “ . . . AACCTTGG . . . ”, the corresponding cigar string is M1 D1 M6, in which M shows matching and D shows deletion. This means that, from the beginning, one character (A) is matched, one character (A) is deleted, and 6 characters (CCTTGG) are continuously matched in the following. As a result of “position of the relative reference genome+one cigar string”, the read sequence data can be completely reduced in case of the reference sequence and the cigar string is better compressed relative to original random characters. For this reason, the ordinary compressor processes the read sequence as “position of the relative reference genome+one cigar string” by virtue of comparison, and then compresses the same.

Two most common technical indicators for measuring compression algorithm performances or efficiencies comprise compression ratio or compression rate; compression/decompression time or compression/decompression speed. Compression ratio=(data size after compression/data size before compression)*100%, compression rate (data size before compression/data size after compression), namely, the compression ratio and the compression rate are the inverse of each other. The compression ratio and compression rate are only in connection with the compression algorithms which can be compared with each other directly, showing better algorithm performance or efficiency when the compression ratio is lower or the compression rate is higher; the compression/decompression time means machine running time required from original data reading to decompression; the compression/decompression speed means data volume that can be processed every unit time averagely. The compression/decompression time and the compression/decompression speed are relevant to the compression algorithm and the used machine environment (including hardware and system software). As a result of this, the compression/decompression time or compression/decompression speeds of various algorithms must be meaningful based on the same machine environment. It is on this premise that the algorithm performance or efficiency is better when the compression/decompression time is shorter and the compression/decompression speed is faster. Besides, an additional reference technical indicator is resource consumption at runtime, mainly a peak value stored by machines. When the compression ratio and compression/decompression time are equivalent, the less storage requirements indicate the better algorithm performance or efficiency.

The comparative research results of the existing gene sequencing data compression methods made by the researchers indicate that the general purpose, reference-free and reference-based compression algorithms have the following problems: 1. the compression ratio can be further decreased; 2. when the relatively better compression ratio is obtained, the algorithm compression/decompression time is relatively long, which makes the time cost become a new problem. Besides, compared with the general purpose and reference-free compression algorithms, the reference-based compression algorithm can generally obtain the better compression ratio. However, for the reference-based compression algorithm, the choice of the reference genome will result in the algorithm performance stability problem, namely, when different reference genomes are selected to process the same target sample data, there may be obvious differences in compression algorithm performance; when the same reference genome selection strategies are applied to processing same and different gene sequencing sample data, there may be obvious differences in compression algorithm performances as well. To be specific, for the reference-based compression algorithm, how to improve the compression ratio and compression performance of the gene sequencing data based on the reference genome has been an urgent technical problem to be solved.

### SUMMARY

The technical problem to be solved by the present invention is to provide a gene sequencing data compression method and decompression method, a system and a computer-readable medium with respect to the above problems of the prior art. The present invention has the advantages of low compression ratio, short compression time and stable compression performance; gene data does not need to be accurately compared, and accordingly, a higher computing efficiency is obtained; the compression rate decreases when the comparison accuracy of the most approximate equal-length gene character sequences CS of the read sequence R is high and the repeated character strings increase.

To solve the above technical problem, the technical solution applied by the present invention is as follows:

On one hand, the present invention provides a gene sequencing data compression method, including the following implementation steps:

A1) traversing a gene sequencing data sample (data) to obtain a read sequence R with a length of Lr;

A2) comparing every read sequence R with the reference genome to obtain the most approximate position p of every read sequence from the reference genome, so as to obtain the most approximate equal-length gene character sequence CS of the read sequence R; coding the read sequence R and the equal-length gene character sequence CS, and then performing reversible computing by means of the reversible function, wherein the output computing results coded by any pair of same characters are identical by virtue of the reversible function; and compressing the most approximate position p of the read sequence R in the reference genome and the reversible computing result that serve as two data streams, and outputting the compressed data streams.

Preferably, step A2) comprises the following detailed steps:

A2.1) traversing the gene sequencing data sample (data) to obtain the read sequence R with the length of Lr;

A2.2) comparing the read sequence R with the reference genome to obtain the most approximate position p thereof from the reference genome, so as to obtain the most approximate equal-length gene character sequence CS of the read sequence R;

A2.3) coding the read sequence R and the equal-length gene character sequence CS, and then performing reversible computing by means of the reversible function, wherein the output computing results coded by any pair of same characters are identical by virtue of the reversible function;

A2.4) compressing the most approximate position p of the read sequence R in the reference genome and the reversible computing result that serve as two data streams, and outputting the compressed data streams;

A2.5) judging whether the read sequence R in the gene sequencing data sample (data) is traversed, if not, jumping to step A2.1); otherwise ending and exiting.

Preferably, XOR computing or bit subtraction is specifically applied for the reversible function.

Preferably, compression in step A2) specifically refers to compression using a statistical model and entropy coding.

On the other hand, the present invention also provides a gene sequencing data decompression method, including the following implementation steps:

B1) traversing gene sequencing data (data) to be decompressed to obtain a read sequence R_{c} to be decompressed;

B2) decompressing and reconstructing every read sequence R_{c} to be decompressed to be a most approximate position p in the reference genome and a reversible computing result CS1 with a length of Lr bit; obtaining a gene character string CS2 with the length of Lr bit in the reference genome according to the most approximate position p in the reference genome; performing reverse computing for the reversible computing result CS1 and the gene character string CS2 by virtue of an inverse function of the reversible function, so as to obtain and output an original read sequence R of the corresponding read sequence R_{c} to be decompressed, wherein the output computing results coded by any pair of same characters are identical by virtue of the reversible computing.

Preferably, step B2) comprises the following detailed steps:

B2.1) traversing gene sequencing data (data_{c}) to be decompressed to obtain the read sequence R_{c} to be decompressed;

B2.2) decompressing and reconstructing the read sequence R_{c} to be decompressed to the most approximate position p in the reference genome and the reversible computing result CS1 with the length of Lr bit;

B2.3) obtaining the gene character string CS2 with the length of Lr bit from the reference genome according to the most approximate position p in the reference genome;

B2.4) performing reverse computing for the reversible computing result CS1 and the gene character string CS2 by virtue of the inverse function of the reversible function, so as to obtain and output the original read sequence R of the corresponding read sequence R_{c} to be decompressed, wherein the output computing results coded by any pair of same characters are identical by virtue of the reversible computing;

B2.5) judging whether the read sequence R_{c} to be decompressed in the gene sequencing data sample (data_{c}) to be decompressed is traversed, if not, jumping to step B2.1); otherwise ending and exiting.

Preferably, an XOR function or a bit subtraction function is specifically applied for the reversible function. An inverse function of the XOR function is the XOR function, and an inverse function of the bit subtraction function is a bit addition function.

Preferably, decompression and reconstruction in step B2) specifically refer to decompression and reconstructing using inverse algorithms of a statistical model and entropy coding.

Besides, the present invention further provides a gene sequencing data decompression system, comprising a computer system, wherein the computer system is programmed to perform the steps of the aforesaid gene sequencing data compression method or the aforesaid gene sequencing data decompression method of the present invention.

Besides, the present invention further provides a computer-readable medium on which a computer program is stored, wherein the computer program enables a computer to perform the steps of the aforesaid gene sequencing data compression method or the aforesaid gene sequencing data decompression method of the present invention.

The present invention has the following advantages:

1. The gene sequencing data compression method of the present invention is the lossless and reference-based gene sequencing data compression method, comprising the steps of comparing a read sequence R with a reference genome to obtain an equal-length gene character sequence CS; coding the read sequence R and the equal-length gene character sequence CS, and then performing reversible computing by means of a reversible function; and compressing a most approximate position p of the read sequence R in the reference genome and the reversible computing result that serve as two data streams, and outputting the compressed data streams. The gene sequencing data compression method is capable of effectively improving the compression ratio of the gene sequencing data, and has the advantages of low compression ratio, short compression time and stable compression performance.

2. Different from using the reference sequence for precise comparison for the gene sequences and then performing data compression in the prior art, in the method of the present invention, gene data does not need to be accurately compared when the read sequence R and the reference genome are compared to obtain the equal-length gene character sequence CS. The computing efficiency increases when the comparison accuracy decreases. Based on this, the compression ratio decreases when the repeated character strings in the reversible computing result increase.

3. According to the method of the present invention, when the read sequence R and the reference genome are compared to obtain the equal-length gene character sequence CS, various gene sequencing data comparison methods may be generally applied to obtaining high efficiency and accuracy of the most approximate equal-length gene character sequence CS of the read sequence R. Based on this, the compression ratio decreases when the compression efficiency increases.

The gene sequencing data decompression method is a reverse method corresponding to the gene sequencing data compression method of the present invention, and has the same advantages as the aforesaid advantages of the gene sequencing data compression method of the present invention, so it will not be repeated here.

### BRIEF DESCRIPTION OF THE DRAWINGS

FIG. 1 is a basic schematic diagram of a compression method in the embodiments of the present invention.

FIG. 2 is a basic schematic diagram of a decompression method in the embodiments of the present invention.

### DETAILED DESCRIPTION

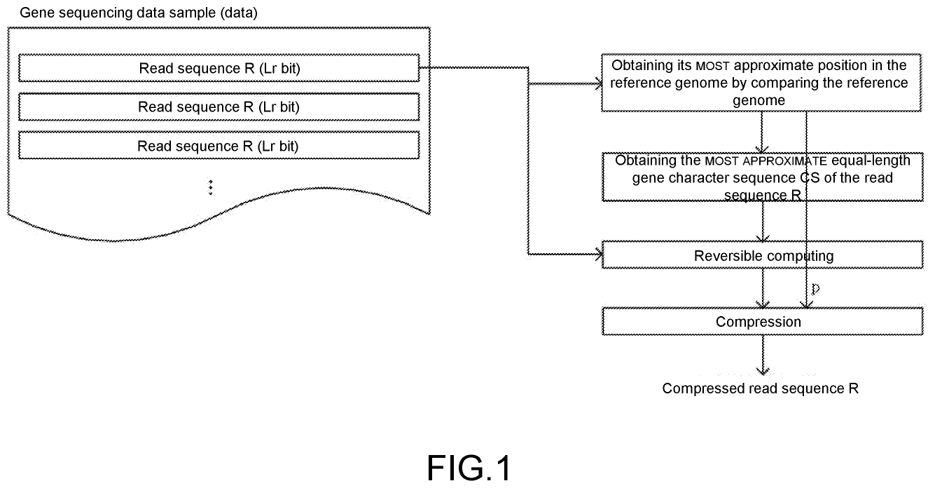

By referring to FIG. 1, the gene sequencing data compression method of this embodiment comprises the following implementation steps:

A1) traversing a gene sequencing data sample (data) to obtain a read sequence R with a length of Lr;

A2) comparing every read sequence R with the reference genome to obtain the most approximate position p of every read sequence from the reference genome, so as to obtain the most approximate equal-length gene character sequence CS of the read sequence R; coding the read sequence R and the equal-length gene character sequence CS, and then performing reversible computing by means of the reversible function, wherein the output computing results coded by any pair of same characters are identical by virtue of the reversible function; and compressing the most approximate position p of the read sequence R in the reference genome and the reversible computing result that serve as two data streams, and outputting the compressed data streams.

According to the gene sequencing data compression method in this embodiment, the compression ratio is further reduced, the compression/decompression time of an algorithm is relatively shorter while a better compression ratio is obtained; the present invention is compatible with algorithms for making comparisons between read sequences and reference genomes.

In this embodiment, step A2) comprises the following detailed steps:

A2.1) traversing the gene sequencing data sample (data) to obtain the read sequence R with the length of Lr;

A2.2) comparing the read sequence R with the reference genome to obtain the most approximate position p thereof from the reference genome, so as to obtain the most approximate equal-length gene character sequence CS of the read sequence R;

A2.3) coding the read sequence R and the equal-length gene character sequence CS, and then performing reversible computing by means of a reversible function, wherein the output computing results coded by any pair of same characters are identical by virtue of the reversible function;

A2.4) compressing the most approximate position p of the read sequence R in the reference genome and the reversible computing result that serve as two data streams, and outputting the compressed data streams;

A2.5) judging whether the read sequence R in the gene sequencing data sample (data) is traversed, if not, jumping to step A2.1); otherwise ending and exiting.

In this embodiment, XOR computing or bit subtraction is specifically applied for the reversible function.

In this embodiment, compression in step A2) specifically refers to compression using a statistical model and entropy coding.

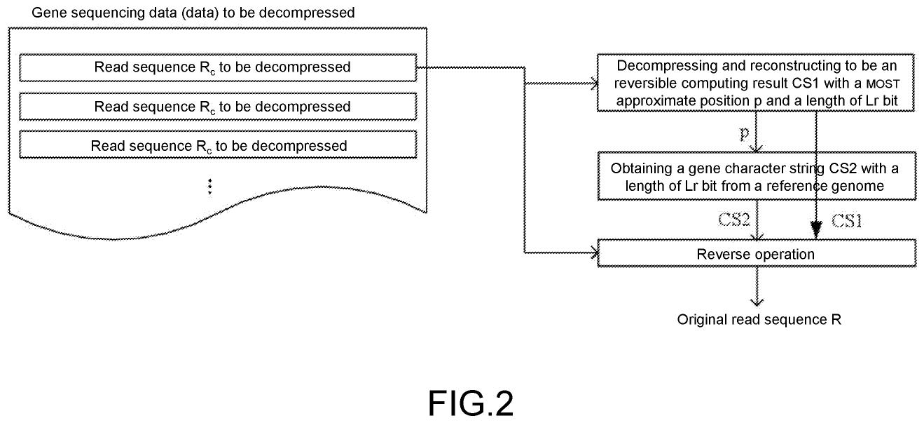

By referring to FIG. 2, the gene sequencing data decompression method of this embodiment comprises the following implementation steps:

B1) traversing gene sequencing data (data) to be decompressed to obtain a read sequence R_{c} to be decompressed;

B2) decompressing and reconstructing every read sequence R_{c} to be decompressed to be a most approximate position p in the reference genome and a reversible computing result CS1 with a length of Lr bit; obtaining a gene character string CS2 with the length of Lr bit in the reference genome according to the most approximate position p in the reference genome; performing reverse computing for the reversible computing result CS1 and the gene character string CS2 by virtue of an inverse function of the reversible function, so as to obtain and output an original read sequence R of the corresponding read sequence R_{c} to be decompressed, wherein the output computing results coded by any pair of same characters are identical by virtue of the reversible computing.

In this embodiment, step B2) comprises the following detailed steps:

B2.1) traversing gene sequencing data (data_{c}) to be decompressed to obtain the read sequence R_{c} to be decompressed;

B2.2) decompressing and reconstructing the read sequence R_{c} to be decompressed to the most approximate position p in the reference genome and the reversible computing result CS1 with the length of Lr bit;

B2.3) obtaining the gene character string CS2 with the length of Lr bit from the reference genome according to the most approximate position p in the reference genome;

B2.4) performing reverse computing for the reversible computing result CS1 and the gene character string CS2 by virtue of the inverse function of the reversible function, so as to obtain and output the original read sequence R of the corresponding read sequence R_{c} to be decompressed, wherein the output computing results coded by any pair of same characters are identical by virtue of the reversible computing;

B2.5) judging whether the read sequence R_{c} to be decompressed in the gene sequencing data sample (data_{c}) to be decompressed is traversed, if not, jumping to step B2.1); otherwise ending and exiting.

An XOR function or a bit subtraction function is specifically applied for the reversible function. An inverse function of the XOR function is the XOR function, and an inverse function of the bit subtraction function is a bit addition function. In this embodiment, XOR computing is specifically applied for the reversible computing. In this embodiment, A, C, G and T gene letters are respectively coded as 00, 01, 10 and 11, for instance, a certain gene letter is A, and a prediction character c is A at the same, an XOR operation result (reversible computing result) of this bit is 00, otherwise the XOR operation result varies according to different input characters; in decompressing, the XOR operation (reverse computing for the inverse function of the XOR function) is performed for the character coding and XOR operation result (reversible computing result) of the prediction character c again, namely, original gene characters can be restored. A, C, G and T gene letters are respectively coded as 00, 01, 10 and 11, which is a preferable streamlined coding way. Besides, other binary coding ways may be applied for reversible conversion between the gene characters, prediction characters and reversible computing results according to the needs. Without doubt, the subtraction may be applied for reversible computing in addition to the XOR computing, and meanwhile the inverse computing of the reversible computing is addition. Meanwhile, the reversible conversion between the gene characters, prediction characters and reversible computing results can be implemented.

In this embodiment, decompression and reconstruction in step B2) specifically refer to decompression and reconstructing using inverse algorithms of a statistical model and entropy coding.

Besides, this embodiment further provides a gene sequencing data decompression system, comprising a computer system, wherein the computer system is programmed to perform the steps of the aforesaid gene sequencing data compression method or the aforesaid gene sequencing data decompression method of the present invention.

Besides, this embodiment further provides a computer-readable medium on which a computer program is stored, wherein the computer program enables a computer to perform the steps of the aforesaid gene sequencing data compression method or the aforesaid gene sequencing data decompression method of the present invention.

The above are only preferred embodiments of the present invention, and the protection scope of the present invention is not limited to the embodiment mentioned above. The technical solutions under the ideas of the present invention fall into the protection scope of the present invention. It should be pointed out that, for an ordinary person skilled in the art, some improvements and modifications without departing from the principle of the present invention shall be deemed as the protection scope of the present invention.

1. A gene sequencing data compression method, comprising the following implementation steps:
A1) traversing a gene sequencing data sample data to obtain a read sequence R with a length of Lr;
A2) comparing every read sequence R with the reference genome to obtain a most approximate position p of every read sequence from the reference genome, so as to obtain a most approximate equal-length gene character sequence CS of the read sequence R;
coding the read sequence R and the equal-length gene character sequence CS, and then performing reversible computing by means of a reversible function, wherein output computing results coded by any pair of same characters are identical by virtue of the reversible function; and
compressing the most approximate position p of the read sequence R in the reference genome and the reversible computing result that serve as two data streams, and outputting the compressed data streams.

2. The gene sequencing data compression method as recited in claim 1, wherein the step A2) comprises the following detailed steps:
A2.1) traversing the gene sequencing data sample data to obtain a read sequence R with the length of Lr;
A2.2) comparing the read sequence R with the reference genome to obtain a most approximate position p thereof from the reference genome, so as to obtain a most approximate equal-length gene character sequence CS of the read sequence R;
A2.3) coding the read sequence R and the equal-length gene character sequence CS, and then performing reversible computing by means of a reversible function, wherein the output computing results coded by any pair of same characters are identical by virtue of the reversible function;
A2.4) compressing the most approximate position p of the read sequence R in the reference genome and the reversible computing result that serve as two data streams, and outputting the compressed data streams;
A2.5) judging whether the read sequence R in the gene sequencing data sample data is traversed, if not, jumping to step A2.1); otherwise ending and exiting.

3. The gene sequencing data compression method as recited in claim 1, wherein a XOR computing or a bit subtraction is specifically applied for the reversible function.

4. The gene sequencing data compression method as recited in claim 1, wherein the compression in step A2) specifically refers to a compression using a statistical model and entropy coding.

5. A gene sequencing data decompression method, comprising the following implementation steps:
B1) traversing gene sequencing data data_{c} to be decompressed to obtain a read sequence R_{c} to be decompressed;
B2) decompressing and reconstructing every read sequence R_{c} to be decompressed to be a most approximate position p in the reference genome and a reversible computing result CS1 with and a length of Lr bit;
obtaining a gene character string CS2 with the length of Lr bit in the reference genome according to the most approximate position p in the reference genome;
performing reverse computing for the reversible computing result CS1 and the gene character string CS2 by virtue of an inverse function of the reversible function, so as to obtain and output an original read sequence R of the corresponding read sequence R_{c} to be decompressed, wherein the output computing results coded by any pair of same characters are identical by virtue of the reversible computing.

6. The gene sequencing data decompression method as recited in claim 5, wherein the step B2) comprises the following detailed steps:
B2.1) traversing gene sequencing data data_{c} to be decompressed to obtain a read sequence R_{c} to be decompressed;
B2.2) decompressing and reconstructing the read sequence R_{c} to be decompressed to a most approximate position p in the reference genome and the reversible computing result CS1 with a length of Lr bit;
B2.3) obtaining a gene character string CS2 with the length of Lr bit from the reference genome according to the most approximate position p in the reference genome;
B2.4) performing reverse computing for the reversible computing result CS1 and the gene character string CS2 by virtue of an inverse function of an reversible function, so as to obtain and output an original read sequence R of the corresponding read sequence R_{c} to be decompressed, wherein the output computing results coded by any pair of same characters are identical by virtue of the reversible computing;
B2.5) judging whether the read sequence R_{c} to be decompressed in the gene sequencing data sample data_{c} to be decompressed is traversed, if not, jumping to step B2.1); otherwise ending and exiting.

7. The gene sequencing data decompression method as recited in claim 5, wherein an XOR function or a bit subtraction function is specifically applied for the reversible function; An inverse function of the XOR function is the XOR function, and an inverse function of the bit subtraction function is a bit addition function.

8. The gene sequencing data decompression method as recited in claim 5, wherein the decompression and reconstruction in step B2) specifically refer to decompression and reconstructing using inverse algorithms of a statistical model and entropy coding.

9. A gene sequencing data decompression system, comprising a computer system, wherein the computer system is programmed to perform the steps of the gene sequencing data compression method as recited in claim 1.

10. A computer-readable medium on which a computer program is stored, wherein the computer program enables a computer to perform the steps of the gene sequencing data compression method as recited in claim 1.

11. The gene sequencing data compression method as recited in claim 2, wherein a XOR computing or a bit subtraction is specifically applied for the reversible function.

12. The gene sequencing data decompression method as recited in claim 6, wherein an XOR function or a bit subtraction function is specifically applied for the reversible function; An inverse function of the XOR function is the XOR function, and an inverse function of the bit subtraction function is a bit addition function.

13. A gene sequencing data decompression system, comprising a computer system, wherein the computer system is programmed to perform the steps of the gene sequencing data compression method as recited in claim 2.

14. A gene sequencing data decompression system, comprising a computer system, wherein the computer system is programmed to perform the steps of the gene sequencing data compression method as recited in claim 3.

15. A gene sequencing data decompression system, comprising a computer system, wherein the computer system is programmed to perform the steps of the gene sequencing data compression method as recited in claim 4.

16. A gene sequencing data decompression system, comprising a computer system, wherein the computer system is programmed to perform the steps of the gene sequencing data decompression method as recited in claim 5.

17. A gene sequencing data decompression system, comprising a computer system, wherein the computer system is programmed to perform the steps of the gene sequencing data decompression method as recited in claim 6.

18. A gene sequencing data decompression system, comprising a computer system, wherein the computer system is programmed to perform the steps of the gene sequencing data decompression method as recited in claim 7.

19. A gene sequencing data decompression system, comprising a computer system, wherein the computer system is programmed to perform the steps of the gene sequencing data decompression method as recited in claim 8.

20. A computer-readable medium on which a computer program is stored, wherein the computer program enables a computer to perform the steps of the gene sequencing data compression method as recited in claim 2.
